(19) United States
(12) Patent Application Publication
(10) Pub. No.: US 2025/0120766 A1
(43) Pub. Date: Apr. 17, 2025

(54) AUTOMATED IDENTIFICATION OF HYPERINTENSIVE APPARENT DIFFUSION COEFFICIENT CLUSTERS FOR MRI-GUIDED LASER INTERSTITIAL THERMAL THERAPY

(71) Applicant: THE JOHNS HOPKINS UNIVERSITY, Baltimore, MD (US)

(72) Inventors: Min Jae KIM, Fairfax, VA (US); Brian HWANG, Baltimore, MD (US); David MAMPRE, Baltimore, MD (US); Joon Yi KANG, Baltimore, MD (US); William Stanley ANDERSON, Towson, MD (US)

(21) Appl. No.: 18/694,612

(22) PCT Filed: Jul. 27, 2022
(86) PCT No.: PCT/US2022/038458

§ 371 Date: Mar. 22, 2024

Related U.S. Application Data
(60) Provisional application No. 63/261,802, filed on Sep. 29, 2021.

(51) Int. Cl. A61B 18/20; A61B 5/055; A61B 18/00; G01R 33/56; G01R 33/563

(52) U.S. Cl. CPC A61B 18/20; A61B 5/055; G01R 33/5608; G01R 33/56341; A61B 2018/00446; A61B 2018/00577; A61B 2018/00779; A61B 2018/00982

(57) ABSTRACT

A method includes receiving a diffusion weight image (DWI), a structural T1 image, or both from a magnetic resonance imaging (MRI) sequence on a patient's brain. The patient suffers from drug-resistant mesial temporal epilepsy (mTLE). The method also includes generating an apparent diffusion coefficient (ADC) map of the patient's brain based at least partially upon the DWI, the structural T1 image, or both. The method also includes segmenting one or more mesial temporal lobe portions from a remainder of the patient's brain based at least partially upon the ADC map. The method also includes identifying one or more clusters to be ablated in the one or more mesial temporal lobe portions.

### CROSS-REFERENCE TO RELATED APPLICATIONS

This application is the national stage entry of International Patent Application No. PCT/US2022/038458, filed on Jul. 27, 2022, and published as WO 2023/055471 A1 on Apr. 6, 2023, which claims the benefit of U.S. Provisional Patent Application Ser. No. 63/261,802, filed on Sep. 29, 2021, which are hereby incorporated by reference herein in their entireties.

### FIELD OF THE DISCLOSURE

The present disclosure relates generally to systems and methods for treating drug-resistant mesial temporal epilepsy (mTLE). More particularly, the present disclosure relates to systems and methods for performing MRI-guided laser interstitial thermal therapy (MRgLiTT) to treat drug-resistant mTLE.

### BACKGROUND OF THE DISCLOSURE

Currently, in the United States, there are an estimated 143,000-191,000 drug-resistant mesial temporal epilepsy (mTLE) patients, whose only viable therapeutic option is surgical intervention. These mTLE patients often experience impaired levels of consciousness during seizures that drastically reduces their quality of life.

MRI-guided laser interstitial thermal therapy (MRgLiTT) is a minimally invasive surgical procedure that is rapidly becoming an alternate first-line surgical option in drug-resistant mTLE. While the MRgLiTT procedure is promising, patients who undergo MRgLiTT experience variable postoperative seizure outcomes, ranging from no seizures to full relapse. This may be partly because the conventional MRgLiTT procedure does not account for individual variability in the size and location of the seizure-generating tissue. Therefore, what is needed is an improved system and method for performing the MRgLiTT procedure.

### SUMMARY

In accordance with an aspect of the present disclosure, a method for planning and/or performing a MRgLiTT procedure to treat a (e.g., drug-resistant) mTLE patient is disclosed. The method includes receiving a diffusion weight image (DWI), a structural T1 image, or both from a magnetic resonance imaging (MRI) sequence on a patient's brain. The patient suffers from drug-resistant mesial temporal epilepsy (mTLE). The method also includes generating an apparent diffusion coefficient (ADC) map of the patient's brain based at least partially upon the DWI, the structural T1 image, or both. The method also includes segmenting one or more mesial temporal lobe portions from a remainder of the patient's brain based at least partially upon the ADC map. The method also includes identifying one or more clusters to be ablated in the one or more mesial temporal lobe portions.

In another embodiment, the method includes receiving a diffusion weight image (DWI), a structural T1 image, or both from a magnetic resonance imaging (MRI) sequence on a patient's brain. The patient suffers from drug-resistant mTLE. The method also includes generating an apparent diffusion coefficient (ADC) map of the patient's brain based at least partially upon the DWI, the structural T1 image, or both. The method also includes segmenting one or more mesial temporal lobe portions from a remainder of the patient's brain based at least partially upon the ADC map. The method also includes identifying one or more clusters in the one or more mesial temporal lobe portions. The method also includes determining one or more characteristics of the one or more clusters. The method also includes determining an ablation volume that includes at least a portion of the one or more clusters. The ablation volume is determined based at least partially upon the characteristics.

A computing system for planning an MRI-guided laser interstitial thermal therapy (MRgLiTT) procedure to treat drug-resistant mesial temporal epilepsy (mTLE) is also disclosed. The computing system includes one or more processors and a memory system. The memory system includes one or more non-transitory computer-readable media storing instructions that, when executed by at least one of the one or more processors, cause the computing system to perform operations. The operations include receiving a diffusion weight image (DWI) from a magnetic resonance imaging (MRI) sequence on a patient's brain. The patient suffers from drug-resistant mTLE. The operations also include generating an apparent diffusion coefficient (ADC) map of the patient's brain based at least partially upon the DWI. The operations also include segmenting a hippocampus, an amygdala, or both from a remainder of the patient's brain based at least partially upon the ADC map. The operations also include identifying a plurality of clusters in the hippocampus, the amygdala, or both based at least partially upon an intra-cluster similarity, a spatial proximity, or both. The clusters include hyperintense clusters. The operations also include determining a plurality of characteristics of the clusters. The characteristics include spatial coordinates of the clusters, a volume of each cluster, an average ADC intensity of voxels in each cluster, a maximum ADC intensity of voxels in each cluster, and a standard error of a mean (SEM) of the ADC intensity of the voxels in each cluster. The operations also include determining an ablation volume that includes at least a portion of the clusters. The ablation volume is determined based at least partially upon the characteristics, a power of a laser generated by an ablation tool to ablate the ablation volume, a duration that the laser contacts the ablation volume, a location of the ablation tool in the patient's brain, and a trajectory of the ablation tool in the patient's brain.

### BRIEF DESCRIPTION OF THE FIGURES

FIG. 1A illustrates a graph showing ADC of total ablated regions vs. surgical outcome, according to an embodiment.

FIG. 1B illustrates a graph showing ADC of ablated regions (e.g., in the amygdala) vs. surgical outcome, according to an embodiment.

FIG. 1C illustrates a graph showing ADC of ablated regions (e.g., in the hippocampus) vs. surgical outcome, according to an embodiment.

FIG. 2 illustrates a visualization of hyperintensive ADC clusters in the hippocampus, according to an embodiment.

FIG. 3 illustrates a graph showing the proportion of ADC cluster volume ablated vs. surgical outcome, according to an embodiment.

FIG. 4 illustrates a flowchart of a method for planning and/or performing a MRgLiTT procedure to treat a (e.g., drug-resistant) mTLE patient, according to an embodiment.

FIG. 5 illustrates an image of an ADC map of the patient's brain, according to an embodiment.

FIG. 6 illustrates an image of the ADC map with the hippocampus segmented from a remainder of the patient's brain, according to an embodiment.

FIG. 7 illustrates an image of the ADC map with a plurality of clusters identified, according to an embodiment.

FIG. 8 illustrates an image of the ADC map showing an ablation volume and an ablation tool, according to an embodiment.

### DETAILED DESCRIPTION OF THE PREFERRED EMBODIMENTS

The presently disclosed subject matter now will be described more fully hereinafter with reference to the accompanying Drawings, in which some, but not all embodiments of the disclosures are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

The present disclosure is directed to systems and methods that can precisely identify regions in the brain for ablation during MRI-guided laser interstitial thermal therapy (MRgLiTT) procedures to treat mesial temporal epilepsy (mTLE). Accounting for the variability in the diseased, seizure-generating tissues in the mesial temporal lobe, and selectively targeting those tissues (rather than a conventional ablation using anatomical landmarks), can lead to better seizure outcomes.

The systems and methods described herein may select seizure-generating regions of the brain for ablation based on pre-operative radiographic data. More particularly, the regions for ablation may be selected based at least partially upon a diffusion tensor imaging (DTI) magnetic resonance imaging (MRI) sequence, which may be captured before the MRgLiTT procedure occurs. The DTI MRI sequence may yield an apparent diffusion coefficient (ADC) map that delineates pathological tissues and may highlight potential hyperintense clusters (e.g., epileptogenic foci) in the mesial temporal lobe. Selectively targeting and ablating the mesial temporal region with pronounced ADC signals may reduce the likelihood of seizure relapse. The systems and methods may also yield a maximum ablation volume to prevent potential cognitive side effects.

As discussed below, the method may include a pre-clinical stage and a clinical stage. During the pre-clinical stage, adjustments may be made to the algorithm's ablation selection paradigm, such as adjusting for volumetric outliers. In addition, the intraoperative parameters (e.g., laser power/energy, ablation tool tip location, etc.) may be predicted to create an ablation volume sufficient to cover the candidate ablation targets. In one embodiment, the MRgLiTT target areas (e.g., the hippocampus and/or the amygdala) may be automatically segmented for the designation of region-specific targets. During the clinical stage, the system and method may be used during the MRgLiTT procedure. The seizure outcomes using the system and method described herein may be statistically compared to the seizure outcomes that do not use the system and method (i.e., the outcomes from conventional systems and methods).

### ADC of the Ablated Regions Between Outcomes

A retrospective study of mTLE patients who underwent MRgLiTT at Johns Hopkins identified two seizure outcome groups. The first group includes patients who had good postoperative seizure outcome (International League Against Epilepsy [ILAE] outcome score I and II). The second group includes patients who did not attain good seizure outcome (ILAE III-VI). The first group had ablation zones that contained significantly higher normalized (Z-scored) ADC signals compared to that of the second group (mean ADC of first group=0.01 vs. mean ADC of second group=−0.29).

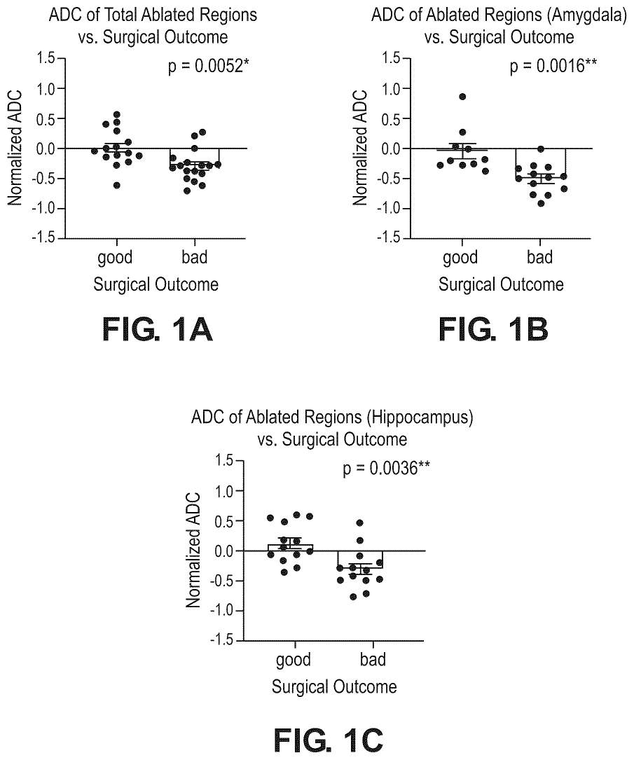

FIG. 1A illustrates a graph showing ADC of total ablated regions vs. surgical outcome, according to an embodiment. FIG. 1B illustrates a graph showing ADC of ablated regions (e.g., in the amygdala) vs. surgical outcome, according to an embodiment. FIG. 1C illustrates a graph showing ADC of ablated regions (e.g., in the hippocampus) vs. surgical outcome, according to an embodiment. The mean (e.g., average) ADC values of the volumes of the total ablated region (FIG. 1A), the ablated region of the amygdala (FIG. 1B), and the ablated region of the hippocampus (FIG. 1C) may be statistically compared with one another based upon the seizure outcomes. Outcomes where no seizures were detected in the 6 months after surgery were classified as good outcomes, and outcomes where one or more seizures were detected in the 6 months after surgery were classified as bad outcomes.

As shown in FIG. 1A, a higher ADC of the total ablated region was associated with good seizure outcomes in univariate analysis (e.g., 0.01±0.08 vs. −0.29±0.06, p=0.0052). As shown in FIGS. 1B and 1C, higher mean ADC intensities of the ablated portion of the hippocampus (e.g., 0.12±0.09 vs. −0.29±0.09, p=0.0036) and of the amygdala (e.g., −0.03±0.12 vs. −0.49±0.07, p=0.0016) were associated with good seizure outcomes in the univariate analysis.

Ablation of Apparent Diffusion Coefficient Hyperintensity Clusters in the Hippocampus During MRgLITT May Improve Seizure Outcomes in mTLE

The regions with hyperintense ADC signals are distributed across the mesial temporal areas as localized clusters. Selectively ablating these clusters may yield improvement in seizure outcomes.

As mentioned above, as one of radiographic measures calculated from DTI, the ADC map delineates pathological tissues and may highlight potential epileptogenic foci in the mesial temporal lobe. Applicant performed tests to determine whether ablation of ADC hyperintensity clusters is associated with better seizure outcome in mTLE patients undergoing MRgLITT.

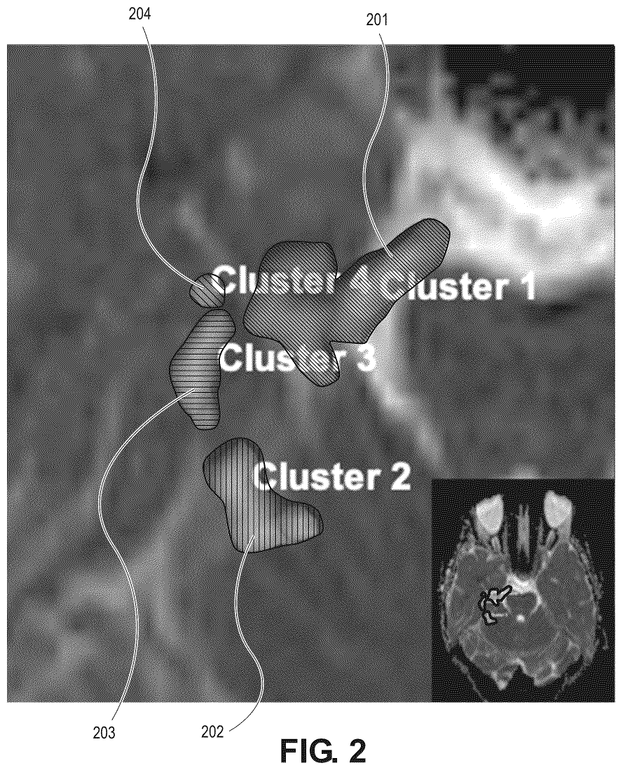

In the test, thirty-three mTLE patients who underwent MRgLiTT were retrospectively studied. A six-month seizure outcome was evaluated using the International League Against Epilepsy (ILAE) outcome scale. Volumes of the hippocampus and amygdala were segmented from the pre-operative ADC map. A machine learning approach was used to identify and segment spatially compact clusters of hyperintense regions. This is shown in FIG. 2. More particularly, FIG. 2 illustrates a visualization of hyperintensive ADC clusters 201-204 in the hippocampus, according to an embodiment. The ablation volume was segmented from the intra-operative magnetization-prepared rapid gradient-echo (MP-RAGE) sequence. Proportions of (1) ADC cluster volume ablated and (2) ADC local maximal peaks of each cluster ablated were correlated with seizure outcome.

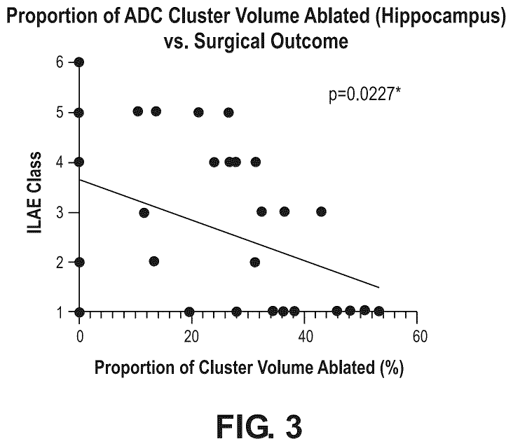

The mean age of patients at surgery was 37.9 years, and the mean follow-up duration was 1.9 years. Twenty-six patients were diagnosed with mesial temporal sclerosis. The proportion of hippocampal ADC cluster volumes ablated correlated with the seizure outcome (e.g., r=−0.402, p<0.05. This is shown in FIG. 3. More particularly, FIG. 3 illustrates a graph showing the proportion of ADC cluster volume ablated vs. surgical outcome, according to an embodiment. The proportion of the peaks of each hippocampal ADC cluster ablated was also significantly associated with seizure outcome (e.g., r=−0.417, p<0.05). For the amygdala, no significant correlation was observed between the proportion of ablated ADC cluster volumes or the peaks of the cluster.

The seizure outcomes in mTLE patients undergoing MRgLITT correlated with the proportion of ADC hyperintensity clusters in the hippocampus that is within the ablation zone. This suggest that ADC clusters in the hippocampus represent epileptogenic tissues that are high yield for laser ablation. Also, machine learning can help identify the high ADC clusters that can enable surgeons to precisely target patient specific epileptogenic foci in the mesial temporal lobe to achieve better seizure outcomes.

### The Method/Workflow

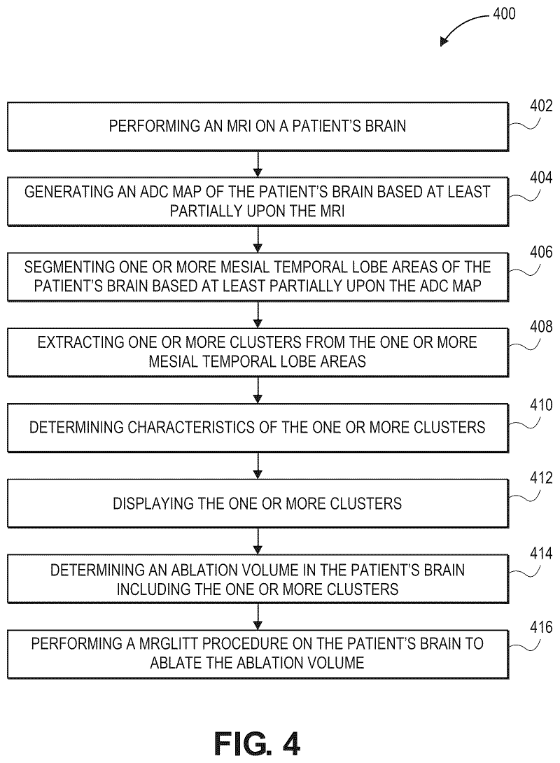

FIG. 4 illustrates a flowchart of a method 400 for planning and/or performing a MRgLiTT procedure to treat a (e.g., drug-resistant) mTLE patient, according to an embodiment. An illustrative order of the method 400 is provided below; however, one or more steps of the method 400 may be performed in a different order, combined, split into sub-steps, repeated, or omitted. One or more steps of the method 400 may be performed by a computing system.

The method 400 may include performing an MRI on a patient's brain, as at 402. More particularly, this may include performing an MRI sequence on the patient's brain to capture/produce a diffusion weight image (DWI) and/or a structural T1 image. The DWI may be used to guide neurosurgeons to identify the trajectory of the tip of an ablation tool during an MRgLiTT procedure.

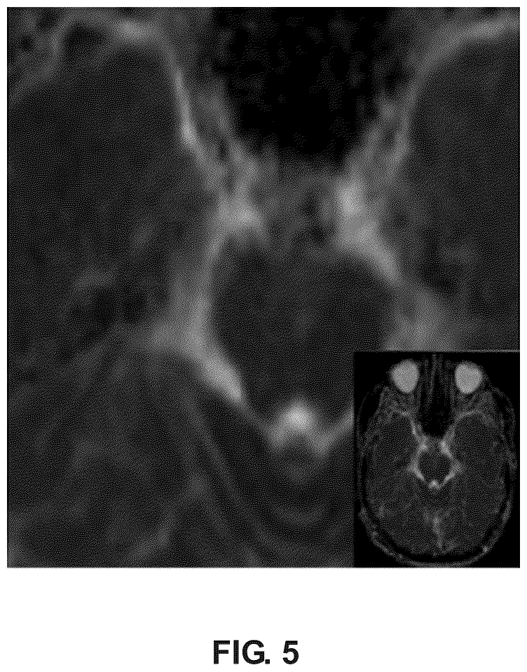

The method 400 may also include generating an ADC map of the patient's brain based at least partially upon the MRI, as at 404. This step may be performed by the computing system. More particularly, this step may include generating the ADC map based at least partially upon the DWI and/or the structural T1 image. FIG. 5 illustrates an image of an ADC map of the patient's brain, according to an embodiment.

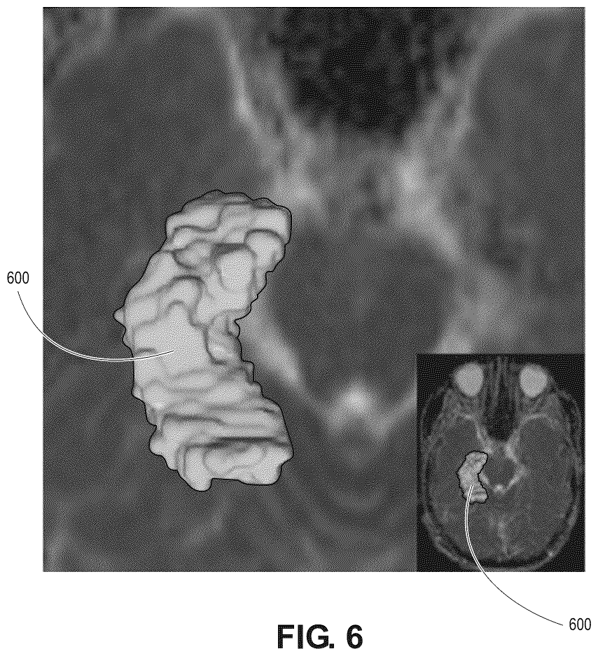

The method 400 may also include segmenting one or more mesial temporal lobe areas from a remainder of the patient's brain based at least partially upon the ADC map, as at 406. This step may be performed by the computing system. The mesial temporal lobe areas (also referred to volumes, portions, or structures) may also or instead be segmented based at least partially upon the DWI and/or the structural T1 image. The mesial temporal lobe areas may be or include the hippocampus, the amygdala, the uncus, the dentate gyrus, the parahippocampal gyrus, or a combination thereof. Extratemporal lobe areas may also be segmented. FIG. 6 illustrates an image of the ADC map with the hippocampus 600 segmented from a remainder of the patient's brain, according to an embodiment.

The method 400 may also include extracting one or more clusters from the one or more mesial temporal lobe areas of the patient's brain, as at 408. This step may be performed by the computing system. More particularly, this step may include identifying and extracting one or more candidate clusters from the one or more segmented mesial temporal lobe areas of the patient's brain based at least partially upon intra-cluster ADC similarity and/or spatial proximity. The extracting in this step does not refer to a physical extraction. The clusters may be or include hyperintense clusters. Hyperintense clusters refer to isolated regions within the segmented mesial temporal lobe areas that include abnormally high relative ADC signals compared to the rest. The ADC signals including/covering the cluster volumes are the local maxima across the rest of the ADC values of the mesial temporal area. While the exact distribution of intra-cluster ADC signals may vary across clusters and patients, the mean magnitude of intra-cluster signals of the hippocampus from normalized ADC map was at minimum 3 times the mean magnitude of ADC values of entire hippocampus from our preliminary study cohort. For example, the clusters may be or include epileptogenic foci. The intra-cluster ADC similarity refers to magnitude of the difference of ADC signals within the same cluster. The spatial proximity refers to a mean pairwise Euclidean distance between two coordinates within same cluster.

The method 400 may also include determining one or more characteristics of the one or more clusters, as at 410. This step may be performed by the computing system. For example, the input(s) into an algorithm run on the computing system may be or include the segmented mesial temporal lobe areas in the ADC map, and the output(s) from the algorithm may be or include one or more characteristics of the clusters. The algorithm may depend at least partially upon the number of clusters and/or the volume of the mesial temporal lobe areas. The characteristics may help the neurosurgeon select and/or prioritize which clusters to ablate.

The characteristics may be or include the spatial coordinates within the ADC map for each cluster. For example, 1=cluster element, and 0=non cluster element. The characteristics may also or instead include the volume of each cluster. The characteristics may also or instead include the average ADC intensity of the voxels of/in each cluster. The characteristics may also or instead include the maximum ADC intensity of the voxels of/in each cluster. The characteristics may also or instead include the standard error of the mean (SEM) of the ADC intensity of the voxels of/in each cluster. Table 1 below provides an example of the output characteristics.

TABLE 1

| Coordinates | | Volume of | Mean | Max | SEM of |
|---|---|---|---|---|---|
| (X, Y, Z, | Cluster | Cluster | ADC of | ADC of | ADC of |
| ADC) | ID | (mm³) | Cluster | Cluster | Cluster |
| 5×4 single | 1 | 28.7001 | 1.6768 | 1.8258 | 0.0507 |
| 105×4 single | 2 | 602.7018 | 2.5857 | 5.5099 | 0.0849 |
| 5×4 single | 3 | 28.7001 | 1.8491 | 2.4244 | 0.1639 |
| 19×4 single | 4 | 2.6309 | 2.6309 | 4.5858 | 0.2171 |
| 33×4 single | 5 | 3.2515 | 3.2515 | 6.4253 | 0.2597 |

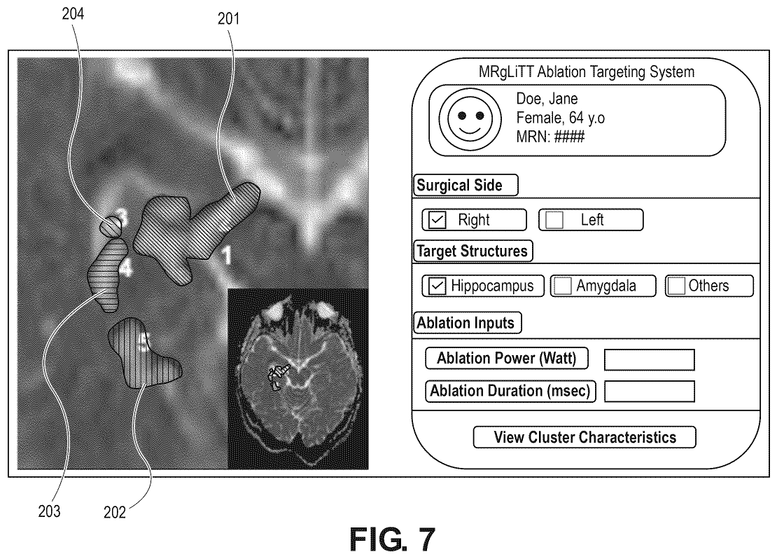

The method 400 may also include displaying the one or more clusters, as at 412. In one embodiment, the MRI image(s), the DWI(s), the structural T1 image(s), the ADC map, the mesial temporal area(s), the clusters, or a combination thereof may be displayed (e.g., using the computing system). FIG. 7 illustrates an image of the ADC map with a plurality of clusters 201-204 identified, according to an embodiment. As described below, the display of the clusters 201-204 may help to guide the doctor during the MRgLiTT procedure.

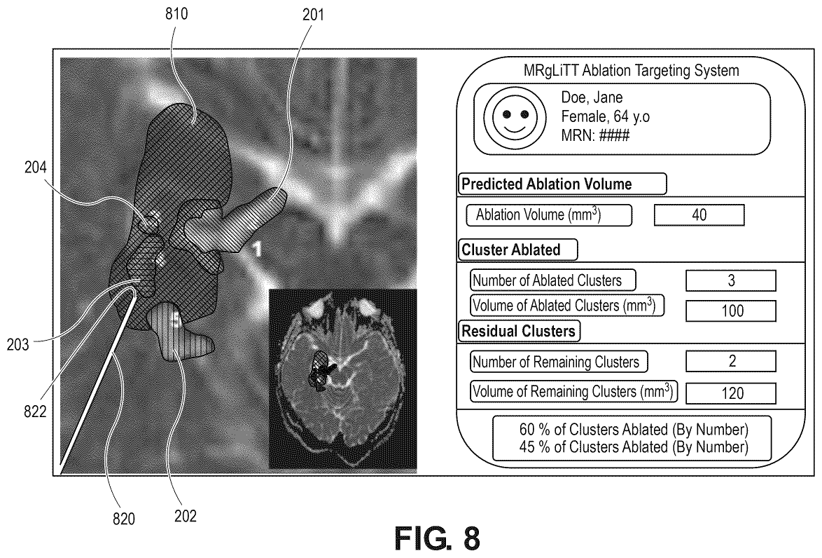

The method 400 may also include determining an ablation volume including at least a portion of the one or more clusters 201-204, as at 414. This step may be performed by the computing system. FIG. 8 illustrates an image of the ADC map showing the ablation volume 810 and the trajectory tip location 822 of the ablation tool 820, according to an embodiment. The ablation volume 810 may depend at least partially upon the characteristics, one or more ablation parameter inputs (e.g., ablation power and/or duration), the trajectory tip location 822, or a combination thereof. The user interface may show the ablation volume 810 and what a laser trajectory would look like going through the clusters 201-204. This may be helpful preoperatively when neurosurgeons are planning trajectory to target as many clusters as possible. Intraoperatively, the user interface may show a real-time burning of the clusters 201-204, allowing the neurosurgeon to confirm that the clusters 201-204 have been ablated. The maximum ablation volume may be determined to prevent potential cognitive side effects.

The method 400 may also include performing a MRgLiTT procedure on the patient's brain, as at 416. More particularly, this may include performing the MRgLiTT procedure to ablate the clusters 201-204 using the ablation tool 820. The MRgLiTT procedure may be performed based at least partly upon the extracted clusters 201-204, the determined ablation volume(s) 810, the displayed clusters 201-204 and/or ablation volume(s) 810, or a combination thereof.

Although the present disclosure has been described in connection with preferred embodiments thereof, it will be appreciated by those skilled in the art that additions, deletions, modifications, and substitutions not specifically described may be made without departing from the spirit and scope of the disclosure as defined in the appended claims.

1. A method, comprising:
receiving a diffusion weight image (DWI), a structural T1 image, or both from a magnetic resonance imaging (MRI) sequence on a patient's brain, wherein the patient suffers from drug-resistant mesial temporal epilepsy (mTLE);
generating an apparent diffusion coefficient (ADC) map of the patient's brain based at least partially upon the DWI, the structural T1 image, or both;
segmenting one or more mesial temporal lobe portions from a remainder of the patient's brain based at least partially upon the ADC map; and
identifying one or more clusters to be ablated in the one or more mesial temporal lobe portions.

2. The method of claim 1, wherein the one or more clusters are identified based at least partially upon an intra-cluster ADC similarity, a spatial proximity, or both.

3. The method of claim 1, further comprising determining one or more characteristics of the one or more clusters.

4. The method of claim 3, wherein the characteristics comprise spatial coordinates of the one or more clusters.

5. The method of claim 3, wherein the characteristics comprise a volume of each cluster.

6. The method of claim 3, wherein the characteristics comprise an average ADC intensity of voxels in each cluster.

7. The method of claim 3, wherein the characteristics comprise a maximum ADC intensity of voxels in each cluster.

8. The method of claim 3, wherein the characteristics comprise a standard error of a mean (SEM) of the ADC intensity of the voxels in each cluster.

9. The method of claim 3, further comprising determining an ablation volume that includes at least a portion of the one or more clusters, wherein the ablation volume is determined based at least partially upon the characteristics.

10. The method of claim 9, wherein the ablation volume is also determined based at least partially upon:
a power of a laser generated by an ablation tool to ablate the ablation volume;
a duration that the laser contacts the ablation volume;
a location of the ablation tool in the patient's brain;
a trajectory of the ablation tool in the patient's brain; or
a combination thereof.

11. A method for planning an MRI-guided laser interstitial thermal therapy (MRgLiTT) procedure to treat drug-resistant mesial temporal epilepsy (mTLE), the method comprising:
receiving a diffusion weight image (DWI), a structural T1 image, or both from a magnetic resonance imaging (MRI) sequence on a patient's brain, wherein the patient suffers from drug-resistant mTLE;
generating an apparent diffusion coefficient (ADC) map of the patient's brain based at least partially upon the DWI, the structural T1 image, or both;
segmenting one or more mesial temporal lobe portions from a remainder of the patient's brain based at least partially upon the ADC map;
identifying one or more clusters in the one or more mesial temporal lobe portions;
determining one or more characteristics of the one or more clusters; and
determining an ablation volume that includes at least a portion of the one or more clusters, wherein the ablation volume is determined based at least partially upon the characteristics.

12. The method of claim 11, wherein the one or more clusters are identified based at least partially upon an intra-cluster similarity, a spatial proximity, or both.

13. The method of claim 11, wherein the characteristics comprise:
spatial coordinates of the one or more clusters;
a volume of each cluster;
an average ADC intensity of voxels in each cluster;
a maximum ADC intensity of voxels in each cluster;
a standard error of a mean (SEM) of the ADC intensity of the voxels in each cluster; or
a combination thereof.

14. The method of claim 11, wherein the ablation volume is also determined based at least partially upon:
a power of a laser generated by an ablation tool to ablate the ablation volume;
a duration that the laser contacts the ablation volume;
a location of the ablation tool in the patient's brain;
a trajectory of the ablation tool in the patient's brain; or
a combination thereof.

15. The method of claim 11, further comprising performing the MRgLiTT procedure to ablate the ablation volume in the patient's brain using an ablation tool.

16. A computing system for planning an MRI-guided laser interstitial thermal therapy (MRgLiTT) procedure to treat drug-resistant mesial temporal epilepsy (mTLE), the computing system comprising:
one or more processors; and
a memory system comprising one or more non-transitory computer-readable media storing instructions that, when executed by at least one of the one or more processors, cause the computing system to perform operations, the operations comprising:
receiving a diffusion weight image (DWI) from a magnetic resonance imaging (MRI) sequence on a patient's brain, wherein the patient suffers from drug-resistant mTLE;
generating an apparent diffusion coefficient (ADC) map of the patient's brain based at least partially upon the DWI;
segmenting a hippocampus, an amygdala, or both from a remainder of the patient's brain based at least partially upon the ADC map;
identifying a plurality of clusters in the hippocampus, the amygdala, or both based at least partially upon an intra-cluster similarity, a spatial proximity, or both, wherein the clusters comprise hyperintense clusters;
determining a plurality of characteristics of the clusters, wherein the characteristics comprise:
spatial coordinates of the clusters;
a volume of each cluster;
an average ADC intensity of voxels in each cluster;
a maximum ADC intensity of voxels in each cluster; and
a standard error of a mean (SEM) of the ADC intensity of the voxels in each cluster; and
determining an ablation volume that includes at least a portion of the clusters, wherein the ablation volume is determined based at least partially upon:
the characteristics;
a power of a laser generated by an ablation tool to ablate the ablation volume;
a duration that the laser contacts the ablation volume;
a location of the ablation tool in the patient's brain; and
a trajectory of the ablation tool in the patient's brain.

17. The computing system of claim 16, wherein the ablation volume is determined to minimize a volume of the patient's brain that is to be ablated that does not comprise the clusters.

18. The computing system of claim 16, further comprising displaying the hyperintense clusters, the ablation volume, or both prior to performing the MRgLiTT procedure.

19. The computing system of claim 16, further comprising displaying the ablation volume and the ablation tool in real-time while the MRgLiTT procedure is being performed.

20. The computing system of claim 16, further comprising providing instructions to a surgeon for how to perform the MRgLiTT procedure to ablate the ablation volume with the ablation tool.
